(19) United States
(12) Patent Application Publication
(10) Pub. No.: US 2026/0209188 A1
(43) Pub. Date: Jul. 23, 2026

(54) COMPOSITION FOR PREVENTING OR TREATING RETINAL DISEASES

(71) Applicants: SEOUL NATIONAL UNIVERSITY HOSPITAL, Seoul (KR); SEOUL NATIONAL UNIVERSITY R&DB FOUNDATION, Seoul (KR)

(72) Inventors: Kyu Hyung Park, Seongnam-si (KR); Kwangsic Joo, Seongnam-si (KR); Seung Woo Choi, Seongnam-si (KR); Gyeong Yun Jeon, Seongnam-si (KR); Minji Kim, Seongnam-si (KR)

(73) Assignees: SEOUL NATIONAL UNIVERSITY HOSPITAL, Seoul (KR); SEOUL NATIONAL UNIVERSITY R&DB FOUNDATION, Seoul (KR)

(21) Appl. No.: 19/433,060

(22) Filed: Dec. 25, 2025

Related U.S. Application Data
(63) Continuation-in-part of application No. PCT/KR2024/005151, filed on Apr. 17, 2024.

(30) Foreign Application Priority Data
Jun. 27, 2023 (KR) 10-2023-0082567

(51) Int. Cl. C07D 261/02; A61K 31/194; A61K 31/341; A61K 31/415; A61K 31/519; A61P 27/02; C07C 61/09; C07D 307/04; C07D 487/04; C07D 487/20

(52) U.S. Cl. CPC C07D 261/02; A61K 31/194; A61K 31/341; A61K 31/415; A61K 31/519; A61P 27/02; C07C 61/09; C07D 307/04; C07D 487/04; C07D 487/20

(57) ABSTRACT

The present specification discloses a novel composition for preventing or treating retinal diseases. In one aspect, compounds in the present invention are extremely effective in protecting cells against the cytotoxicity of all-trans-retinal, inhibiting cellular aging, and inhibiting intracellular reactive oxygen species, and thus can be used in therapeutic agents for retinal diseases such as hereditary macular degeneration and age-related macular degeneration.

### CROSS-REFERENCE TO RELATED APPLICATION(S)

This application is a continuation-in-part of PCT/KR2024/005151 filed Apr. 17, 2024, which claims priority to and the benefit of Korean Patent Application No. 10-2023-0082567 filed Jun. 27, 2023 the disclosure of which is incorporated herein by reference in its entirety.

### TECHNICAL FIELD

This specification describes a novel composition for preventing or treating retinal diseases.

Meanwhile, the present application was supported by the following national research and development project.
[National research and development project that has supported the present invention]
[Project Number]1711166085
[Grant Number]2020R1A2C2011189
[Implementing Agency] Ministry of Science and ICT
[Research Management Agency] National Research Foundation of Korea
[Project Name] Individual Basic Research (Ministry of Science and ICT)
[Project Title] Drug Repurposing Focusing on Genetic Pathways in Hereditary Retinal Diseases
[Contribution Rate] ½
[Organization] Seoul National University
[Research Period] Mar. 1, 2020 to Feb. 28, 2023
[National research and development project that has supported the present invention]
[Project Number]1711147389
[Grant Number]2019R1C1C1009345
[Implementing Agency] Ministry of Science and ICT
[Research Management Agency] National Research Foundation of Korea
[Project Name] Individual Basic Research (Ministry of Science and ICT) (R&D)
[Project Title] Identification of Pathological Mechanisms of Retinal Diseases through Correlation Analysis of Blood Microbiomes and Human Genomes
[Contribution Rate] ½
[Organizer] Seoul National University Bundang Hospital
[Research Period] Mar. 1, 2019 to Feb. 28, 2022

### BACKGROUND

ABCA4 is present in the photoreceptor cells of the retina and serves as a conduit protein that receives external light stimulation and transports all-trans-retinal, which is essential for the visual cycle, from the photoreceptor cells to the retinal pigment epithelium cells. Diseases caused by genetic mutations in ABCA4, one of the three most common causative genes of hereditary retinal diseases, result in premature blindness, especially at a young age. Diseases caused by ABCA4 genetic mutations include Stargardt disease, cone dystrophy, and cone-rod dystrophy. Due to genetic mutations in ABCA4, all-trans-retinal accumulates in retinal epithelial cells, and this accumulates as a photooxidant in the form of an all-trans-retinal dimer, A2E, through an oxidative mechanism. This toxic substance is not easily degraded and is difficult to excrete, causing a toxic reaction in retinal photoreceptor cells, leading to cell death.

Age-related macular degeneration is the most common cause of blindness in people over 60 worldwide and is a representative disease caused by aging. Age-related macular degeneration may be classified into early dry, late wet, and late atrophic forms. However, there is currently no therapeutic agent for treating the early dry form or for preventing blindness from progressing to the late atrophic form in advanced macular degeneration. Macular degeneration is well known to share similarities with ABCA4-associated retinal diseases in terms of genetics and pathogenesis, and in particular, the excessive accumulation of lipofuscin within the retinal pigment epithelium (RPE) during aging and the visual cycle has been identified as a critical pathophysiological factor. The bisretinoid component N-retinylidene-N-retinylethanolamine (A2E), a representative constituent of lipofuscin, induces phototoxicity and inflammatory responses, leading to impaired RPE cell function and cell death, which in turn promotes drusen formation and structural alterations in the macular region.

Meanwhile, only one therapeutic agent for hereditary retinal diseases is currently known, which is a drug targeting the RPE65 gene. However, only a very small number of patients are eligible for this therapeutic agent, and for most hereditary retinal diseases, no treatment is available, leaving patients to simply watch as they progress to blindness.

Accordingly, the present inventors have made efforts to discover a drug that removes or reduces the cytotoxicity and photooxidation of all-trans-retinal, and as a result, they have confirmed that the compounds of the present invention not only have an excellent cytoprotective effect against cytotoxicity caused by all-trans-retinal and an inhibitory effect against cell aging caused by hydrogen peroxide, but can also effectively suppress the increase in intracellular reactive oxygen species caused by all-trans-retinal and reduce the intracellular reactive oxygen species level to almost below the level of normal cells to which all-trans-retinal is not added, thereby completing the present invention.

### SUMMARY OF THE INVENTION

An object of the present invention is, in one aspect, to provide a novel composition for preventing, ameliorating, or treating retinal diseases.

### TECHNICAL FIELD

In order to achieve the above-described object, the present invention provides a composition for preventing, ameliorating, or treating retinal diseases, which includes as an active ingredient one or more compounds represented by the following General Formulas 1 to 5.

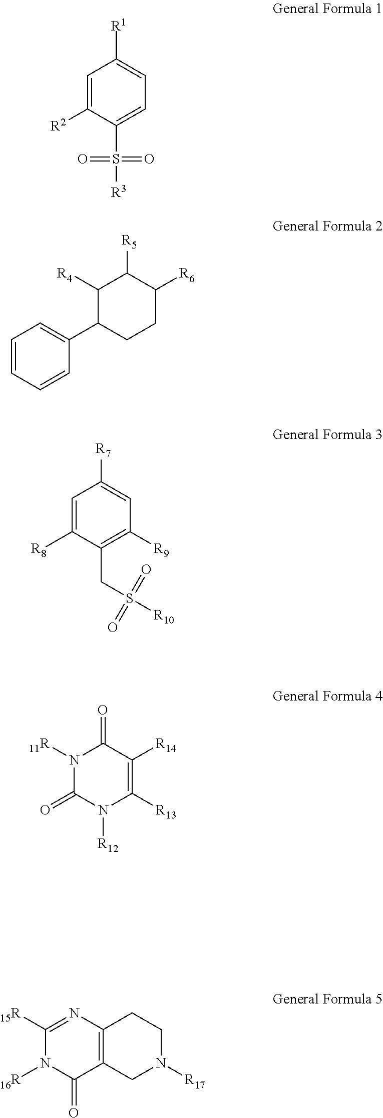

In one exemplary embodiment, R1 to R17 of General Formulas 1 to 5 may each be a substituent selected from the group consisting of a hydrogen, a deuterium, a halogen atom, a hydroxyl group, a cyano group, an amino group, an amido group, a nitro group, a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30)aryl (C1-C30) alkyl group, an alkoxy group, an aryloxy group, a silyl group, a substituted or unsubstituted (C2-C30) alkenyl group, a substituted or unsubstituted (C2-C30) cycloalkenyl group, a substituted or unsubstituted (C1-C30) heteroalkenyl group, a substituted or unsubstituted (C2-C30) alkynyl group, a substituted or unsubstituted (C6-C30) aryl group, a substituted or unsubstituted (C2-C30) heteroaryl group, an acyl group, a carboxylic acid, an ether, an ester, a nitrile, an isonitrile, a sulfanyl group, a sulfinyl group, a sulfonyl group, a phosphino group, and combinations thereof.

In one exemplary embodiment, R13 and R14 may be present independently or may be fused to form a ring.

In one exemplary embodiment, the retinal disease may be one or more selected from the group consisting of hereditary macular degeneration, age-related macular degeneration, myopic macular degeneration, and macular degeneration caused by inflammation.

In one exemplary embodiment, the hereditary macular degeneration may be one or more selected from the group consisting of retinitis pigmentosa, vitelliform macular dystrophy, Stargardt disease, X-linked retinoschisis, and cone dystrophy.

In one exemplary embodiment, the macular degeneration caused by inflammation may be macular degeneration caused by Behcet's uveitis.

In one exemplary embodiment, the compound may have one or more effects of cell death inhibition, cell aging inhibition, or intracellular reactive oxygen species inhibition.

In one exemplary embodiment, the composition may be one or more of a pharmaceutical composition, a quasi-drug composition, and a health functional food composition.

### Advantageous Effects

In one aspect, the compounds of the present invention have excellent cytoprotective effects against cytotoxicity caused by all-trans-retinal, inhibitory effects against cell aging, and inhibitory effects against intracellular reactive oxygen species, and thus can be used as drugs that can treat and inhibit the progression of retinal diseases such as hereditary macular degeneration and age-related macular degeneration.

### BRIEF DESCRIPTION OF THE DRAWINGS

FIGS. 1A and 1B show the results of treating RPE-1, a retinal pigment epithelial cell line, with various concentrations of all-trans-retinal (ATRL).

FIG. 2 shows the selection of compounds that satisfy lead likeness, the Lipinski rule, and the Ghose filter.

FIGS. 3A to 3C show the real-time cytoprotective effects of the final candidate compounds against all-trans-retinal cytotoxicity.

FIG. 4 shows the cytotoxicity of the final candidate compounds themselves.

FIGS. 5 and 6 show the inhibitory effects against cellular senescence of the final candidate compounds.

FIG. 7 shows the reactive oxygen species inhibition activity of compound #17, one of the final candidate compounds.

FIG. 8 presents the quantitative analysis of the proportion of SA-β-gal-positive cells under H₂O₂-induced oxidative stress conditions, demonstrating that co-treatment with compound #17 restores the proportion of SA-β-gal-positive cells to the control level.

FIG. 9 provides microscopic images visualizing the degree of cellular senescence through SA-β-gal staining, in which the number of blue-stained senescent cells increases in the H₂O₂-treated group, whereas in the compound #17 co-treated group the staining intensity and the number of positive cells are reduced and recovered to a level comparable to the control.

FIG. 10 relates to the in vivo efficacy evaluation of compound #17.

FIG. 11 exemplifies the photoreceptor-protective effect of compound #17 in a photodamage model; histological analysis based on H&E staining shows marked thinning of the outer nuclear layer (ONL) and total retinal thickness and disorganized cellular arrangement in the PBS-treated group, whereas the compound #17-treated group maintains ONL thickness and total retinal thickness and structurally preserves photoreceptor cells.

FIG. 12 concerns the retinal protective effect following administration of the liposome-formulated compound #17 eye drops.

FIG. 13 presents in vivo RNA-seq data showing that compound #17 upregulates the expression of FABP5 (Fatty Acid Binding Protein 5) and HSPB1 (Heat Shock Protein Beta-1).

FIG. 14 relates to immunofluorescence analysis visually demonstrating the restoration of HSPB1 protein expression under oxidative stress.

FIG. 15 shows Western blot analysis confirming at the molecular level that compound #17 stabilizes the functional chaperone form by modulating the phosphorylation status of HSPB1.

FIG. 16 is a graph showing PPAR (Peroxisome proliferator-activated receptor α) and ESR (Estrogen receptor β) reporter-based luciferase activity according to the treatment concentration of compound #17; luciferase activity in both PPAR (left) and ESR (middle) reporters decreases in a dose-dependent manner as the concentration of SOR-217 increases, with the greatest inhibitory effect observed in the 100 μM treatment group. In addition, luciferase activity under control (DMSO), negative control, and positive control conditions was compared, and the compound #17-treated group selectively inhibited reporter activity without nonspecific cytotoxicity.

FIG. 17 shows a comparison of mean organ-specific pathological toxicity grades in a non-GLP single-dose toxicity study.

### DETAILED DESCRIPTION

The present invention is described in detail.

The present invention, in one aspect, relates to a composition for preventing, ameliorating, or treating retinal diseases, including as an active ingredient one or more compounds represented by the following General Formulas 1 to 5:

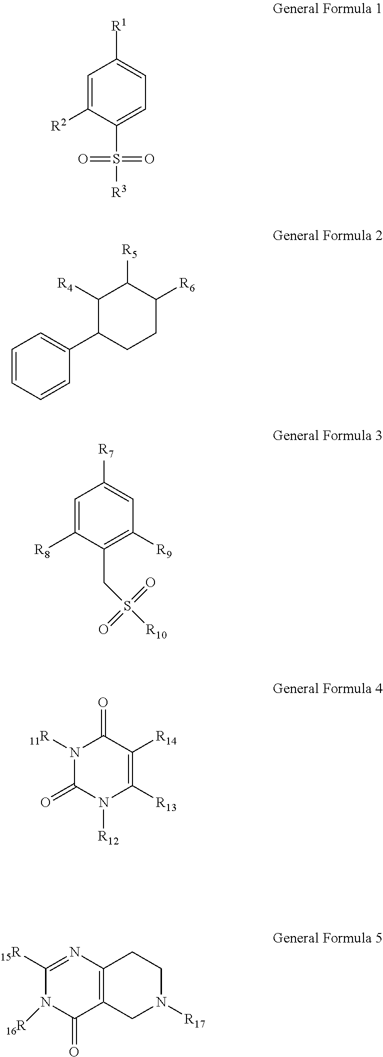

In one exemplary embodiment, R1 to R3 of General Formula 1 may each be a substituent selected from the group consisting of a hydrogen, a deuterium, a halogen atom, a hydroxyl group, a cyano group, an amino group, an amido group, a nitro group, a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, an alkoxy group, an aryloxy group, a silyl group, a substituted or unsubstituted (C2-C30) alkenyl group, a substituted or unsubstituted (C2-C30) cycloalkenyl group, a substituted or unsubstituted (C1-C30) heteroalkenyl group, a substituted or unsubstituted (C2-C30) alkynyl group, a substituted or unsubstituted (C6-C30) aryl group, a substituted or unsubstituted (C2-C30) heteroaryl group, an acyl group, a carboxylic acid, an ether, an ester, a nitrile, an isonitrile, a sulfanyl group, a sulfinyl group, a sulfonyl group, a phosphino group, and combinations thereof.

In one exemplary embodiment, R1 may be a substituent selected from the group consisting of a hydrogen, a deuterium, a halogen atom, and combinations thereof, R2 may be a substituent selected from the group consisting of a hydroxyl group, a cyano group, an amino group, an amido group, a nitro group, an alkoxy group, an aryloxy group, a silyl group, an acyl group, a carboxylic acid group, an ether group, an ester group, a nitrile, an isonitrile, a sulfanyl group, a sulfinyl group, a sulfonyl group, a phosphino group, and combinations thereof, and R3 may be a substituent selected from the group consisting of a substituted or unsubstituted (C1-C30) heteroalkyl group, a substituted or unsubstituted (C2-C30) heterocycloalkyl group, a substituted or unsubstituted (C1-C30) heteroalkenyl group, a substituted or unsubstituted (C2-C30) heteroaryl group, and combinations thereof.

In one exemplary embodiment, R1 may be a halogen atom, R2 may be a cyano group, and R3 may be a substituted or unsubstituted (C2-C30) heterocycloalkyl group. For example, General Formula 1 may be Chemical Formula 1, but is not limited thereto:

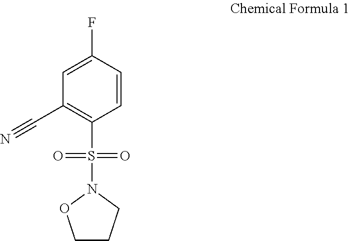

In one exemplary embodiment, R4 to R6 of General Formula 2 may each be a substituent selected from the group consisting of a hydrogen, a deuterium, a halogen atom, a hydroxyl group, a cyano group, an amino group, an amido group, a nitro group, a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, an alkoxy group, an aryloxy group, a silyl group, a substituted or unsubstituted (C2-C30) alkenyl group, a substituted or unsubstituted (C2-C30) cycloalkenyl group, a substituted or unsubstituted (C1-C30) heteroalkenyl group, a substituted or unsubstituted (C2-C30) alkynyl group, a substituted or unsubstituted (C6-C30) aryl group, a substituted or unsubstituted (C2-C30) heteroaryl group, an acyl group, a carboxylic acid, an ether, an ester, a nitrile, an isonitrile, a sulfanyl group, a sulfinyl group, a sulfonyl group, a phosphino group, and combinations thereof.

In one exemplary embodiment, R4 may be a substituent selected from the group consisting of a hydrogen, a deuterium, a substituted or unsubstituted (C1-C30) alkyl group, and combinations thereof, and R5 or R6 may each be a substituent selected from the group consisting of a hydroxyl group, a cyano group, an amino group, an amido group, a nitro group, an alkoxy group, an aryloxy group, a silyl group, an acyl group, a carboxylic acid, an ether, an ester, a nitrile, an isonitrile, a sulfanyl group, a sulfinyl group, a sulfonyl group, a phosphino group, and combinations thereof.

In one exemplary embodiment, R4 may be a substituted or unsubstituted (C1-C5) alkyl group, and R5 or R6 may each be a carboxylic acid or an ester. For example, General Formula 2 may be Chemical Formula 2, but is not limited thereto:

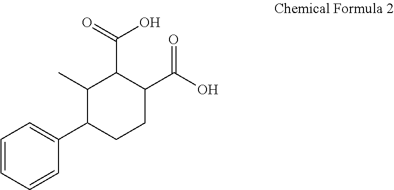

In one exemplary embodiment, R7 to R10 of General Formula 3 may each be a substituent selected from the group consisting of a hydrogen, a deuterium, a halogen atom, a hydroxyl group, a cyano group, an amino group, an amido group, a nitro group, a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, an alkoxy group, an aryloxy group, a silyl group, a substituted or unsubstituted (C2-C30) alkenyl group, a substituted or unsubstituted (C2-C30) cycloalkenyl group, a substituted or unsubstituted (C1-C30) heteroalkenyl group, a substituted or unsubstituted (C2-C30) alkynyl group, a substituted or unsubstituted (C6-C30) aryl group, a substituted or unsubstituted (C2-C30) heteroaryl group, an acyl group, a carboxylic acid, an ether, an ester, a nitrile, an isonitrile, a sulfanyl group, a sulfinyl group, a sulfonyl group, a phosphino group, and combinations thereof.

In one exemplary embodiment, R7 to R9 may each be a substituent selected from the group consisting of a hydrogen, a deuterium, a substituted or unsubstituted (C1-C30) alkyl group, and combinations thereof, and R10 may be a substituent selected from the group consisting of a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, a substituted or unsubstituted (C1-C30) heteroalkenyl group, a substituted or unsubstituted (C2-C30) heteroaryl group, and combinations thereof.

In one exemplary embodiment, R7 to R9 may each be a substituted or unsubstituted (C1-C30) alkyl group, and R10 may be a substituent selected from the group consisting of a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, and combinations thereof. For example, General Formula 3 may be Chemical Formula 3, but is not limited thereto:

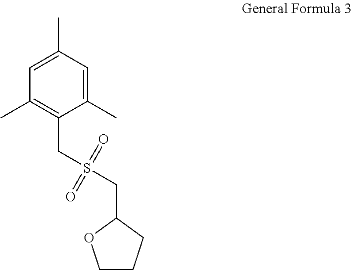

In one exemplary embodiment, R11 to R14 of General Formula 4 may each be a substituent selected from the group consisting of a hydrogen, a deuterium, a halogen atom, a hydroxyl group, a cyano group, an amino group, an amido group, a nitro group, a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, an alkoxy group, an aryloxy group, a silyl group, a substituted or unsubstituted (C2-C30) alkenyl group, a substituted or unsubstituted (C2-C30) cycloalkenyl group, a substituted or unsubstituted (C1-C30) heteroalkenyl group, a substituted or unsubstituted (C2-C30) alkynyl group, a substituted or unsubstituted (C6-C30) aryl group, a substituted or unsubstituted (C2-C30) heteroaryl group, an acyl group, a carboxylic acid, an ether, an ester, a nitrile, an isonitrile, a sulfanyl group, a sulfinyl group, a sulfonyl group, a phosphino group, and combinations thereof, and R13 and R14 may be present independently or may be fused to form a ring.

In one exemplary embodiment, R11 may be a substituent selected from the group consisting of a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, a substituted or unsubstituted (C2-C30) alkenyl group, a substituted or unsubstituted (C2-C30) cycloalkenyl group, a substituted or unsubstituted (C1-C30) heteroalkenyl group, a substituted or unsubstituted (C2-C30) alkynyl group, a substituted or unsubstituted (C6-C30) aryl group, a substituted or unsubstituted (C2-C30) heteroaryl group, and combinations thereof, R12 may be a substituent selected from the group consisting of a hydrogen, a deuterium, a substituted or unsubstituted (C1-C30) alkyl group, and combinations thereof, and R13 and R14 may be fused to form a substituted or unsubstituted ring.

In one exemplary embodiment, R11 may be a substituent selected from the group consisting of a substituted or unsubstituted (C2-C30) cycloalkenyl group, a substituted or unsubstituted (C3-C30) cycloalkyl group, a substituted or unsubstituted (C2-C30) heterocycloalkyl group, and combinations thereof, R12 may be a hydrogen or a deuterium, and R13 and R14 may be fused to form a substituted or unsubstituted (C2-C30) heterocycle. For example, General Formula 4 may be Chemical Formula 4, but is not limited thereto.

In one exemplary embodiment, R11 may be a substituted or unsubstituted (C6-C30) aromatic ring.

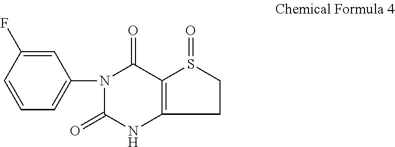 [SMILES: O=c1[nH]c2c(c(=O)n1-c1cccc(F)c1)S(=O)CC2]

In one exemplary embodiment, R15 to R17 of General Formula 5 may each be a substituent selected from the group consisting of a hydrogen, a deuterium, a halogen atom, a hydroxyl group, a cyano group, an amino group, an amido group, a nitro group, a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, an alkoxy group, an aryloxy group, a silyl group, a substituted or unsubstituted (C2-C30) alkenyl group, a substituted or unsubstituted (C2-C30) cycloalkenyl group, a substituted or unsubstituted (C1-C30) heteroalkenyl group, a substituted or unsubstituted (C2-C30) alkynyl group, a substituted or unsubstituted (C6-C30) aryl group, a substituted or unsubstituted (C2-C30) heteroaryl group, an acyl group, a carboxylic acid, an ether, an ester, a nitrile, an isonitrile, a sulfanyl group, a sulfinyl group, a sulfonyl group, a phosphino group, and combinations thereof.

In one exemplary embodiment, R15 may be a substituent selected from the group consisting of a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, a substituted or unsubstituted (C1-C30) heteroalkenyl group, a substituted or unsubstituted (C2-C30) heteroaryl group, and combinations thereof, R16 may be a substituent selected from the group consisting of a hydrogen, a deuterium, a substituted or unsubstituted (C1-C30) alkyl group, and combinations thereof, and R17 may be a substituent selected from the group consisting of a substituted or unsubstituted (C1-C30) alkyl group, a substituted or unsubstituted (C3-C30) cycloalkyl group, a (C1-C30) heteroalkyl group, a substituted or unsubstituted (C2-C30) heterocycloalkyl group, an amino group, an amido group, a nitro group, a carboxylic acid, an ester, and combinations thereof.

In one exemplary embodiment, R15 may be a substituted or unsubstituted (C2-C30) heterocycloalkyl group, R16 may be a hydrogen or a deuterium, and R17 may be a substituent selected from the group consisting of a substituted or unsubstituted (C1-C30) alkyl group, a substituted or unsubstituted (C3-C30) cycloalkyl group, an amino group, an amido group, a nitro group, a carboxylic acid, an ester, and combinations thereof. For example, General Formula 5 may be Chemical Formula 5, but is not limited thereto:

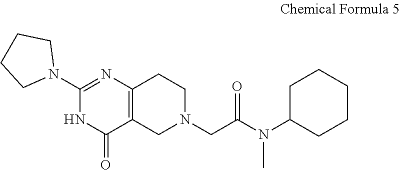

In one exemplary embodiment, the compound represented by [Chemical Formula 1] may be named 5-fluoro-2-(isoxazolidin-2-ylsulfonyl)benzonitrile.

In one exemplary embodiment, the compound represented by [Chemical Formula 2] may be named 3-methyl-4-phenylcyclohexane-1,2-dicarboxylic acid.

In one exemplary embodiment, the compound represented by [Chemical Formula 3] may be named 2-(((2,4,6-trimethylbenzyl)sulfonyl)methyl)tetrahydrofuran.

In one exemplary embodiment, the compound represented by [Chemical Formula 4] may be named 3-(3-fluorophenyl)-6,7-dihydrothieno[3,2-d]pyrimidine-2,4(1H,3H)-dione 5-oxide.

In one exemplary embodiment, the compound represented by [Chemical Formula 5] may be named N-cyclohexyl-N-methyl-2-(4-oxo-2-(pyrrolidin-1-yl)-3,5,7,8-tetrahydropyrido[4,3-d]pyrimidin-6(4H)-yl)acetamide.

In one exemplary embodiment, the composition may include the compound at a concentration of 0.1 μM or more, 10 μM or more, 25 μM or more, 50 μM or more, or 100 μM or more, or 1000 μM or less, 750 μM or less, 500 μM or less, 450 μM or less, 400 μM or less, 350 μM or less, or 300 μM or less. For example, the composition may include the compound at a concentration of 0.1 M to 1000 μM or 10 μM to 500 μM, and when the compound is included at a concentration of less than 0.1 μM, the composition may have poor cell death inhibition, cell senescence inhibition, or intracellular reactive oxygen species inhibition, or when the compound is included at a concentration exceeding 1000 μM, it may exhibit high cytotoxicity.

In one exemplary embodiment, the retinal disease may be one or more selected from the group consisting of hereditary macular degeneration, age-related macular degeneration, myopic macular degeneration, and macular degeneration caused by inflammation, but is not limited thereto.

In one exemplary embodiment, the hereditary macular degeneration may be one or more selected from the group consisting of retinitis pigmentosa, vitelliform macular dystrophy, Stargardt disease, X-linked retinoschisis, and cone dystrophy.

In one exemplary embodiment, the macular degeneration caused by inflammation may be macular degeneration caused by Behcet's uveitis

In one exemplary embodiment, the compound may inhibit cell death induced by all-trans-retinal, thereby exhibiting cytoprotective effects against cytotoxicity induced by all-trans-retinal. The compound includes compounds of General Formulas 1 to 5 as well as derivatives and analogs thereof.

In one exemplary embodiment, the compound may exhibit inhibitory activity against cellular aging induced by hydrogen peroxide. The compound may include compounds of General Formulas 1 to 5, including derivatives and analogs thereof.

In one exemplary embodiment, the compound may reduce intracellular reactive oxygen species levels due to all-trans-retinal. The compound may include compounds of General Formulas 1 to 5, including derivatives and analogs thereof.

In one exemplary embodiment, the composition may be one or more of a pharmaceutical composition, a quasi-drug composition, and a health functional food composition.

In one exemplary embodiment, the pharmaceutical composition may be formulated into a unit dosage form suitable for administration to a patient's body according to a conventional method and administered. Suitable dosage forms for this purpose include parenterally administered preparations such as injections or topical preparations. Injectable dosage forms are preferably isotonic aqueous solutions or suspensions. However, diluents or excipients such as commonly used fillers, bulking agents, binders, wetting agents, disintegrants, and surfactants may also be used. The preparation of the present invention thus prepared may be administered parenterally, for example, directly to a specific site, according to a conventional method. In this case, the composition may be injected using a syringe. It should be understood that the actual dosage of the active ingredient should be determined in light of various relevant factors, such as the administration route and the patient's weight, age, and sex.

In one exemplary embodiment, the pharmaceutical composition may be formulated for oral administration. The oral formulation may be provided in the form of a tablet, capsule, granule, powder, film-coated tablet, hard or soft gelatin capsule, suspension, or solution, and may further include, if necessary, pharmaceutically acceptable diluents, excipients, binders, disintegrants, glidants, lubricants, coating agents, flavoring agents, stabilizers, or enteric coating materials. Preferably, in order to prevent the active ingredient from being inactivated by acid and/or enzymatic degradation in the gastrointestinal tract or by first-pass metabolism, the formulation may be prepared as an enteric formulation, a sustained- or controlled-release formulation, or an oral delivery system for targeted tissue delivery. The oral dose may be determined in consideration of factors such as the patient's weight, age, severity of disease, dosing frequency, and half-life, and may be administered once or more times per day. The oral formulation according to the present invention may be administered alone or in combination with other pharmaceutical agents, and the dosing schedule may be adjusted within a therapeutically acceptable range.

In one exemplary embodiment, the pharmaceutical composition may be formulated as an ophthalmic eye-drop formulation for topical instillation to the eye of a subject. The eye-drop formulation may be provided as a pharmaceutically acceptable aqueous solution, suspension, or liposome- or nanoparticle-based dispersion, and may further include, if necessary, isotonic agents, viscosity-modifying agents, preservatives, osmotic pressure regulators, lubricants, or surfactants. The eye drops may be prepared to remain stable at room temperature or under refrigerated storage conditions and may be instilled once or more times daily at an appropriate frequency depending on the severity of the condition. In addition, the pharmaceutical composition may further include a penetration enhancer, membrane-permeability enhancer, or sustained-release carrier (e.g., liposomes, micelles, polymeric nanoparticles, etc.) so as to facilitate efficient delivery to the retinal pigment epithelium layer.

In one exemplary embodiment, the quasi-drug composition may be added as is or used together with other quasi-drugs or quasi-drug ingredients, and may be used appropriately according to a conventional method. The mixing amount of the active ingredient may be appropriately determined depending on the intended use. The ingredients included in the quasi-drug composition may include, in addition to the active ingredient, ingredients commonly used in quasi-drug compositions, and may include, for example, an abrasive, a wetting agent, a binder, a foaming agent, a sweetener, a preservative, a medicinal ingredient, a flavoring agent, a coloring agent, a solvent, a whitening agent, a solubilizer, or a pH adjuster. The quasi-drug composition may be prepared in any formulation commonly prepared in the art.

In one exemplary embodiment, the health functional food composition may include a food additive acceptable from a food science perspective, and may further include suitable carriers, excipients, and diluents commonly used in the manufacture of foods. In addition to the above, the composition may further include various nutrients, vitamins, electrolytes, flavoring agents, coloring agents, pectic acid and salts thereof, alginic acid and salts thereof, organic acids, protective colloid thickeners, pH adjustors, stabilizers, preservatives, glycerin, alcohol, carbonating agents used in carbonated beverages, and the like.

In another aspect, the present invention relates to a method for preventing, ameliorating, or treating retinal diseases by administrating an effective amount of one or more of the compounds represented by General formulas 1 to 5 to a subject.

In another aspect, the present invention relates to the use of one or more of the compounds represented by General formulas 1 to 5 for the preparation of a composition for preventing, ameliorating, or treating retinal diseases.

In another aspect, the present invention relates to the use of any one or more of the compounds represented by General formulas 1 to 5 for the preparation of a pharmaceutical composition for preventing or treating retinal diseases.

In another aspect, the present invention relates to the use of any one or more of the compounds represented by General formulas 1 to 5 for the preparation of a quasi-drug composition for preventing, ameliorating, or treating retinal diseases.

In another aspect, the present invention relates to the use of any one or more of the compounds represented by General formulas 1 to 5 for the preparation of a health food composition for preventing or ameliorating retinal diseases.

In another aspect, the present invention relates to the use of any one or more of the compounds represented by General formulas 1 to 5 for preventing, ameliorating, or treating retinal diseases.

In another aspect, the present invention relates to the non-therapeutic or therapeutic use of any one or more of the compounds represented by General formulas 1 to 5.

Hereinafter, the present invention will be described in more detail through examples and the like. These examples are intended solely to illustrate the present invention, and it will be apparent to those skilled in the art that the scope of the present invention is not to be construed as being limited by these examples.

### Working Examples

### Example 1 Compound Screening

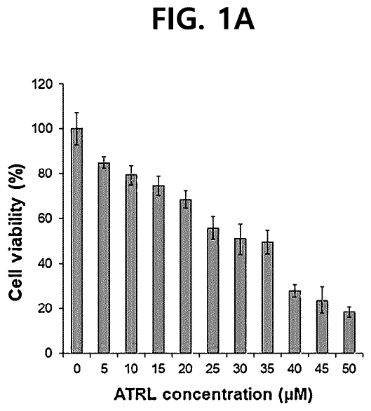

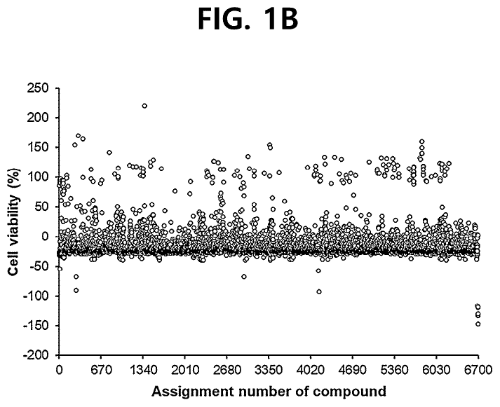

As a result of treating the RPE-1 cell line, a retinal pigment epithelial cell line, with various concentrations of all-trans-retinal (ATRL), a significant decrease was observed in RPE-1 cell viability as the concentration of all-trans-retinal increased (FIGS. 1A and 1B).

Based on the above-described results, a viability test was performed on 6,696 compounds (from a representative compound library of the Korea Chemical Bank) using all-trans-retinal at a concentration of 30 μM. The highest cell viability was 219%, and the top 197 compounds that significantly increased cell viability compared to the control were selected. The top six compounds are listed in Table 1, and the 1,364th compound showed a 219% increase in cell viability compared to the control (Table 1).

TABLE 1
Cell viability of the top six compounds

| | | | | Tested | |
|---|---|---|---|---|---|
| | | Concen- | | concen- | |
| | | tration | Volume | tration | % |
| No | Position | (mM) | (μl) | (mM) | viability |
| 1364 | RPL-D1-000191-D03 | 5.2 | 5 | 0.25 | 219.159082 |
| 306 | RPL-D1-000177-B11 | 5.1 | 5 | 0.24 | 169.055534 |
| 384 | RPL-D1-000178-H10 | 5.2 | 5 | 0.25 | 163.741893 |
| 5795 | RPL-D1-000246-C07 | 5.4 | 5 | 0.26 | 159.466456 |
| 250 | RPL-D1-000177-B04 | 5.2 | 5 | 0.25 | 154.022018 |
| 3362 | RPL-D1-000216-B03 | 5.2 | 5 | 0.25 | 153.229059 |

### Example 2 Structural Analysis of Compounds and Alignment Based on Drug Likeness Criteria (in Silico Analysis)

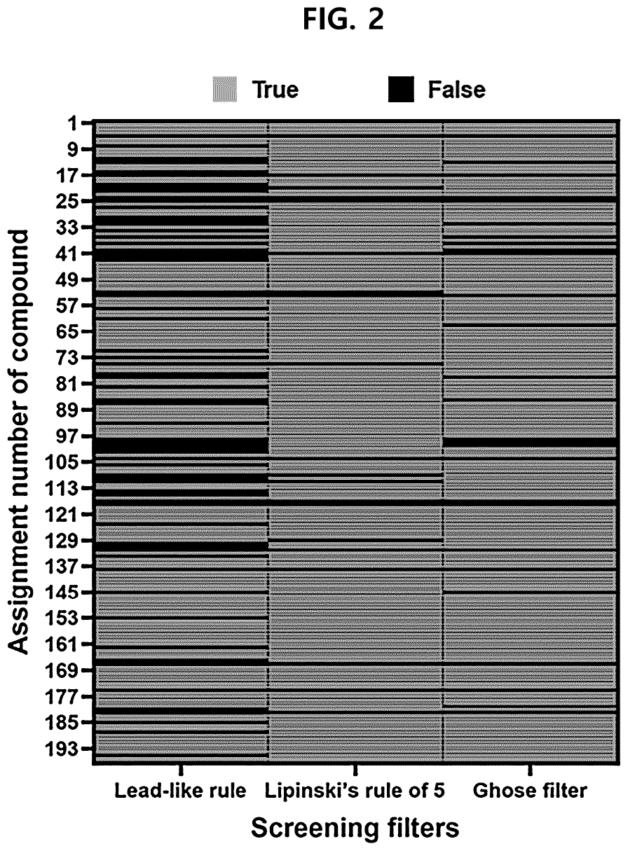

Drug likeness was calculated using ChemoAxon (Table 2), and compounds satisfying lead likeness, Lipinski rule, and Ghose filter were selected (FIG. 2). The proportion of candidate compounds that showed 3, 2, 1, and 0 violations of the three above-mentioned virtual screening filter criteria was 7.1%, 8.1%, 19.8%, and 65.0%, respectively. Structural analysis of the candidate compounds showed that 1) they were compounds with high hydrophobicity that could easily penetrate lipid membranes or remain in the membrane while operating and 2) they had aromatic groups such as benzene rings, suggesting a potential role as a radical scavenger. Thirty compounds were likely not radical scavengers and likely had the possibility of specific binding. A total of 46 compounds were selected, including 34 compounds that were thought to be radical scavengers, among those that satisfied all drug likeness criteria.

TABLE 2
Drug likeness criteria

| | | Lipinski's | |
|---|---|---|---|
| | | Rule of | |
| | | Five | Ghose |
| Property | Lead likeness | (4 of 4) | Filter |
| MW | <=450 | <=500 | 160 <= x <= 480 |
| HB donor count | <=5 | <=5 | |
| HB acceptor count | <=8 | <=10 | |
| LogD (pH 7.4) | −4 <= x <= 4 | | |
| Ring count | <=4 | | |
| Rotatable bond | <=10 | | |
| count | | | |
| LogP | | <=5 | −0.4 <= x <= 5.6 |
| Refractivity | | | 40 <= x <= 130 |
| Atom count | | | 20 <= x <= 70 |
| References: | Teague, S. L. | Adv. Drug | J. Comb. Chem., |
| | et al., Angew. | Deliv. Rev., | 1, 55 (1999). |
| | Chem. Int. Ed. | 23, 3 | |
| | 1999, 38, 3743. | (1997). | |

### Example 3 Selection of Final Compound Using Real-Time Live-Cell Imaging Technology

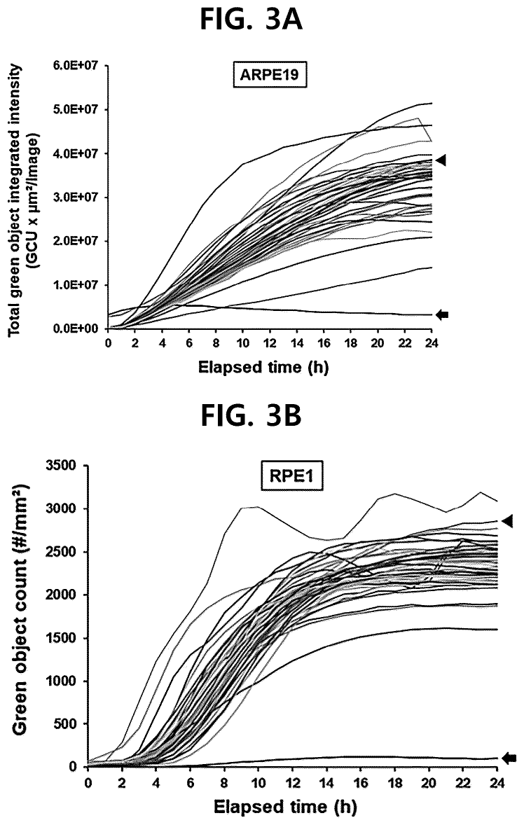

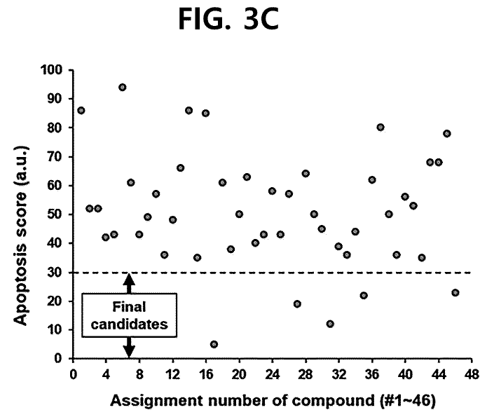

To determine the real-time cytoprotective effects of the final candidate compounds against all-trans-retinal cytotoxicity, ARPE-19 and RPE-1 cells were treated with all-trans-retinal at a concentration of 30 μM. The cells were then treated with the 46 candidate compounds selected in Example 2. Real-time cell viability was measured using Incucyte for 24 hours (FIGS. 3A and 3B). The viability ranks for each cell line were combined and designated an apoptosis score. The five final candidate compounds were then sorted in ascending order of apoptosis scores (i.e., descending order of cell viability) to select the final five candidates (FIG. 3C).

### Example 4 Confirmation of Structure and Cytotoxicity of Five Final Candidate Compounds

The structures of the five final candidate compounds selected through real-time live cell imaging technology performed in Example 3 are shown below.

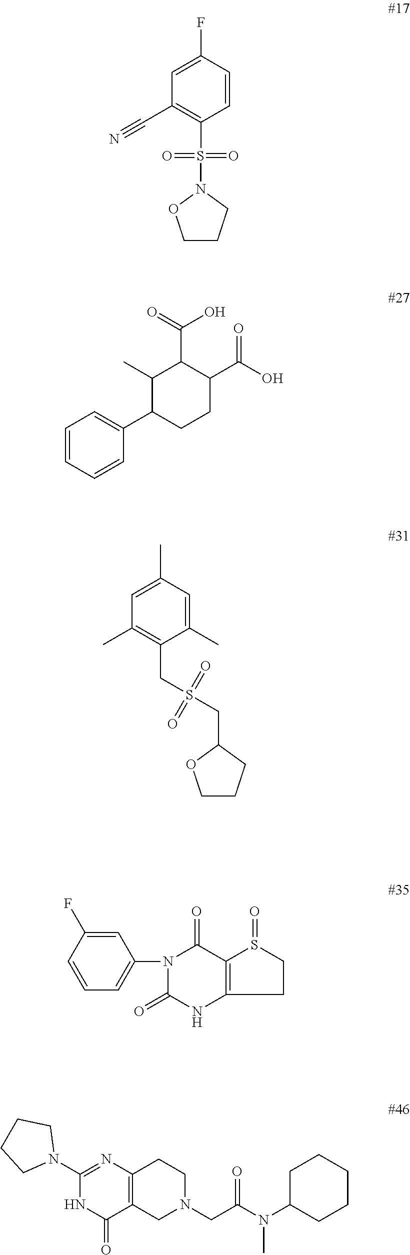 [SMILES: N#Cc1cc(F)ccc1S(=O)(=O)N1CCCO1 ; CC1C(c2ccccc2)CCC(C(=O)O)C1C(=O)O ; Cc1cc(C)c(CS(=O)(=O)CC2CCCO2)c(C)c1 ; O=c1[nH]c2c(c(=O)n1-c1cccc(F)c1)S(=O)CC2 ; CN(C(=O)CN1CCc2nc(N3CCCC3)[nH]c(=O)c2C1)C1CCCCC1]

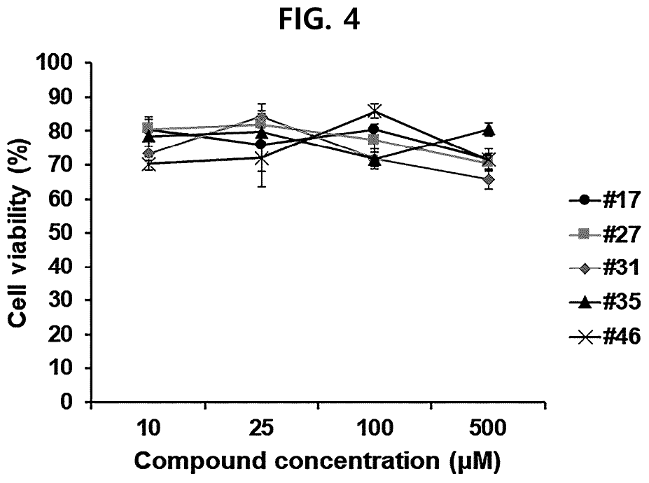

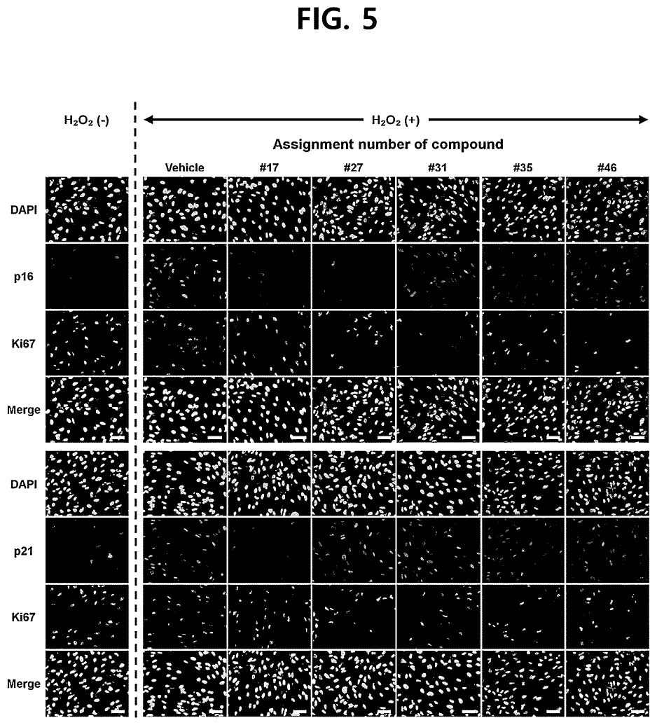

To confirm the cytotoxicity of the final candidate compounds themselves, RPE-1 cells were treated with the candidate compounds at various concentrations (10, 25, 100, 500 μM) and cultured for 24 hours. As a result, relatively good cell viability was confirmed even at high concentrations of the compounds (FIG. 4), confirming that the candidate compounds themselves had minimal toxicity. Compared to the control group, there was no difference in the amount of apoptosis-related factors (such as caspase-9) when treated with candidate compounds, and there were no particular morphological changes in the cells. Bulk RNA-sequencing did not show any significant inflammation or increased expression of apoptosis-related RNAs.

### Example 5 Cellular Aging Analysis with Five Final Candidate Drugs

It is known that premature senescence occurs when retinal pigment epithelial cells are treated with low, sub-cytotoxic concentrations of hydrogen peroxide (H₂O₂). Using this, 100 μM H₂O₂ was applied to ARPE-19 cells, which were then treated with the candidate compounds to determine their senescence inhibition effects.

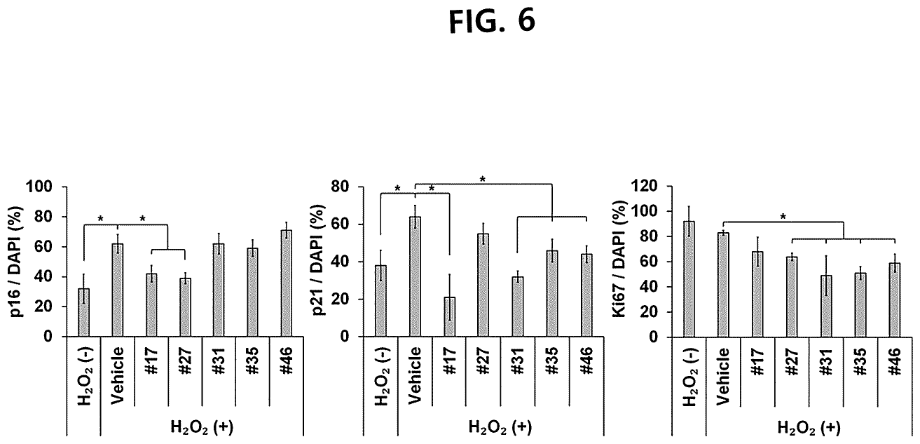

As shown in FIGS. 5 and 6, the expression of p16 and p21, representative senescence markers, was increased in ARPE-19 cells after hydrogen peroxide treatment. A decrease in p16 was observed with compounds #17 and #27, while a decrease in p21 was observed with compounds #17, #31, #35, and #46.

The representative proliferation marker, ki67, showed a decrease in expression in all candidate compound treatment groups. However, the decrease was less pronounced in compound #17, and there was no statistically significant difference compared to the group treated with a negative control substance (vehicle).

Considering these results, compound #17 was determined to have the strongest cytoprotective and senescence inhibition effects, and further experiments were conducted on compound #17.

### Example 6 Confirmation of Reactive Oxygen Species Inhibitory Effect of Compound #17

When all-trans-retinal was applied to RPE-1 cells, an increase in intracellular reactive oxygen species was observed. To confirm the intracellular reactive oxygen species inhibitory effect of compound #17, a DCFDA assay was performed.

When compound #17 was added to cells, it effectively inhibited the increase in intracellular reactive oxygen species induced by all-trans-retinal, reducing intracellular reactive oxygen species levels to levels almost below those of normal cells without all-trans-retinal.

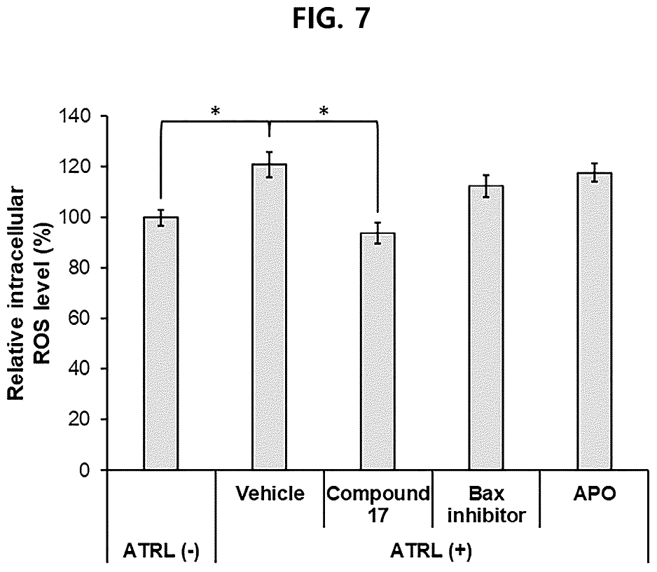

Compared to Bax inhibitor, an anti-apoptotic protein that suppresses intracellular reactive oxygen species accumulation, and apocynin (APO), an antioxidant that inhibits NADPH oxidase, it was confirmed that compound #17 exhibited significantly superior reactive oxygen species inhibition activity (FIG. 7).

In addition, to evaluate the anti-senescence effect of compound #17 in RPE1 cells, a senescence-associated β-galactosidase (SA-R-gal) staining assay was performed. Premature senescence was induced by treating RPE1 cells with H₂O₂ to generate oxidative stress, followed by pretreatment with compound #17 (10 μM) for 24 hours, and the proportion of SA-β-gal-positive cells was quantitatively analyzed. The control group (CON) was cultured without any treatment, and the anti-senescence effect was evaluated by comparing the H₂O₂-only treatment group with the H₂O₂+compound #17 co-treatment group.

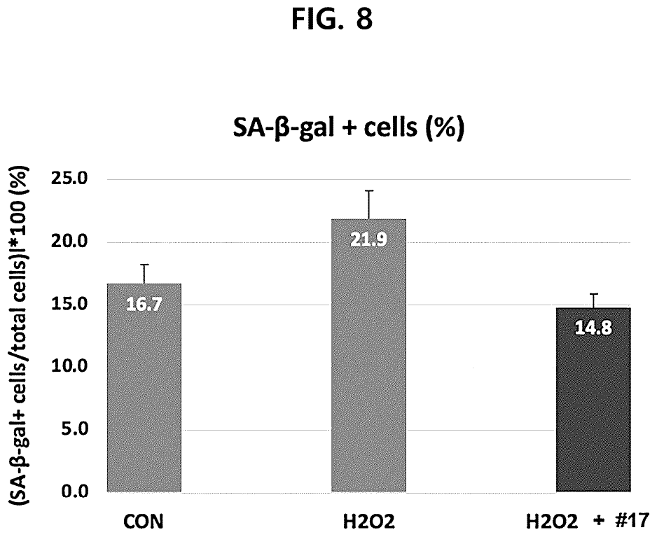

As shown in FIG. 8, the proportion of SA-β-gal-positive cells was 16.7% in the control group, whereas it increased to 21.9% in the H₂O₂-only treatment group, confirming that oxidative stress significantly induced cellular senescence. In contrast, the H₂O₂+compound #17 co-treatment group exhibited a decreased proportion of SA-R-gal-positive cells of 14.8%, indicating recovery to a level below that of the control.

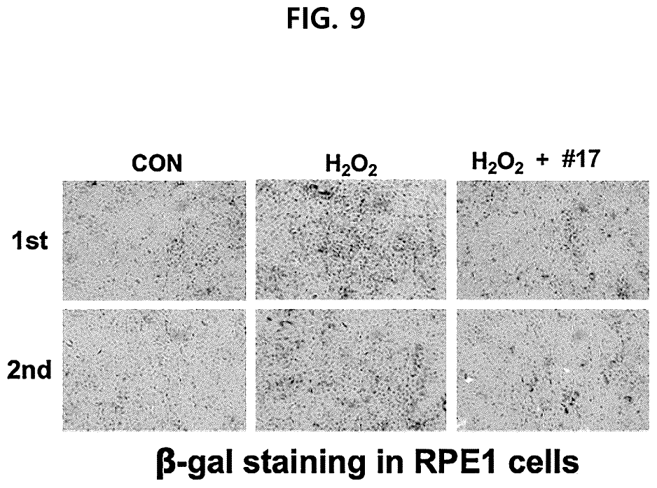

Furthermore, microscopic images in FIG. 9 demonstrated a marked increase in the number and staining intensity of blue-stained SA-β-gal-positive cells in the H₂O₂-treated group compared to the control, whereas the H₂O₂+compound #17 co-treatment group showed a substantial reduction in blue staining, displaying a pattern similar to that of the control. This trend was consistently observed across two independent experiments (1st and 2nd). Collectively, these results demonstrate that compound #17 effectively suppresses oxidative stress-induced premature senescence of RPE cells.

### Example 7 In Vivo Efficacy Evaluation of Compound #17: Reduction of Lipofuscin and Protection of Photoreceptors in a Light-Induced Retinal Damage Model

A2E-lipofuscin is a major component of aging pigments and accumulates within retinal cells in the form of yellow deposits. The outer nuclear layer (ONL) of the retina is the layer where the nuclei of photoreceptors are located and is therefore closely associated with photoreceptor function; accordingly, ONL thickness is widely used as an important biomarker to evaluate photoreceptor health and the progression of retinal degenerative diseases. The optic nerve head is the region where the optic nerve originates from the eye and connects to the brain, serving as the pathway through which the electrical signals converted from light stimuli received by photoreceptors are transmitted to the brain. Therefore, the ONL thickness surrounding the optic nerve head is a key indicator reflecting whether photoreceptor function is preserved, and changes in ONL thickness in this region provide critical information for determining retinal dysfunction and the severity of disease.

ABCA4/RDH8 double-knockout (−/−) C57BL/6 male mice (8 weeks old) were exposed to 12,000 lux white light for 2 hours to establish a retinal degeneration model characterized by excessive A2E accumulation mimicking human Stargardt disease. Compound #17 was intraperitoneally administered at a dose of 10 mg/kg three times per week for a total of 8 weeks, consisting of 4 weeks before and 4 weeks after light exposure.

Additional experiments confirmed that comparable efficacy was achieved even when compound #17 was administered for only 2 or 4 weeks before and after light exposure. The experimental groups consisted of a normal control group, a PBS-treated group after light exposure, and a compound #17-treated group after light exposure.

Following completion of dosing, the eyeballs were enucleated and subjected to fundus imaging, optical coherence tomography (OCT), and retinal histological analyses. The thickness of the outer nuclear layer (ONL) was measured at 400-μm intervals up to 1,600 μm in both the superior and inferior directions from the optic nerve head, and the mean value was calculated from three repeated measurements at each point. In addition, systemic toxicity was evaluated by monitoring body weight, behavioral abnormalities, mortality, and histopathological examination of major organs.

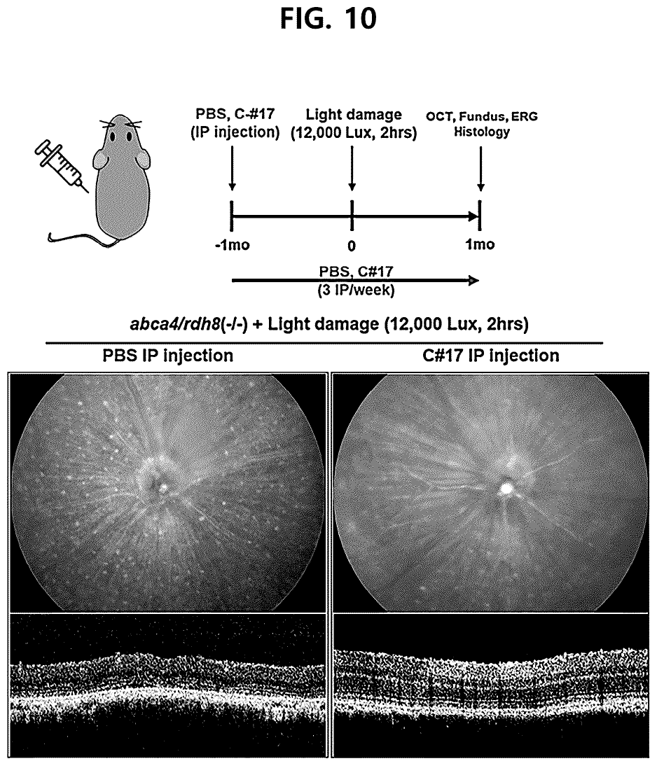

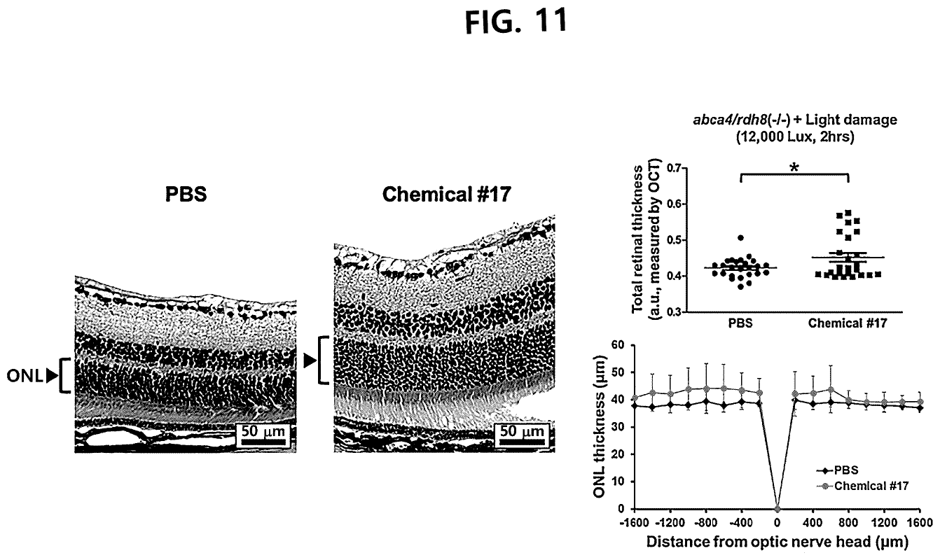

As shown in FIG. 10, the PBS-treated group exhibited a marked accumulation of lipofuscin following light damage, whereas lipofuscin accumulation was effectively suppressed in the compound #17-treated group (p<0.001). Histological examination further demonstrated the disappearance of excessive lipofuscin deposition and normalization of RPE morphology in the compound #17-treated group. In addition, as shown in FIG. 11, the compound #17-treated group maintained significantly greater total retinal thickness and ONL thickness compared with the PBS-treated group, and comparable recovery effects were observed across both central and peripheral retinal regions. Notably, the protective effect was more pronounced in areas closer to the optic nerve head, thereby demonstrating that compound #17 confers photoreceptor protection across the entire retina.

Furthermore, no changes in body weight, behavioral abnormalities, or mortality were observed during the 4-week dosing period, and no nonspecific toxicity findings were detected in major organs such as the liver and kidney, nor in ocular tissues. Collectively, these results indicate that compound #17 is a promising drug candidate exhibiting both potent photoreceptor-protective efficacy and excellent safety.

### Example 8 Retinal Protective Effect Following Administration of Liposome-Formulated Compound #17 Eye Drops

In this experiment, a liposomal eye-drop formulation containing 0.1% compound #17 was prepared and administered five times daily for 4 weeks. The liposomal eye drops maintained stability for at least 3 days under 4° C. storage conditions and were designed as an encapsulated formulation for localized ocular delivery. Compound #17 also exhibited plasma stability for more than 20 hours under room-temperature ex vivo conditions, confirming the feasibility of stable drug delivery. A liposomal eye-drop formulation without compound #17 was used as the control. Retinal protective effects were evaluated by measuring the thickness of the outer nuclear layer (ONL) using OCT imaging and by assessing A2E accumulation and retinal atrophy using FAF (Fundus Autofluorescence) imaging.

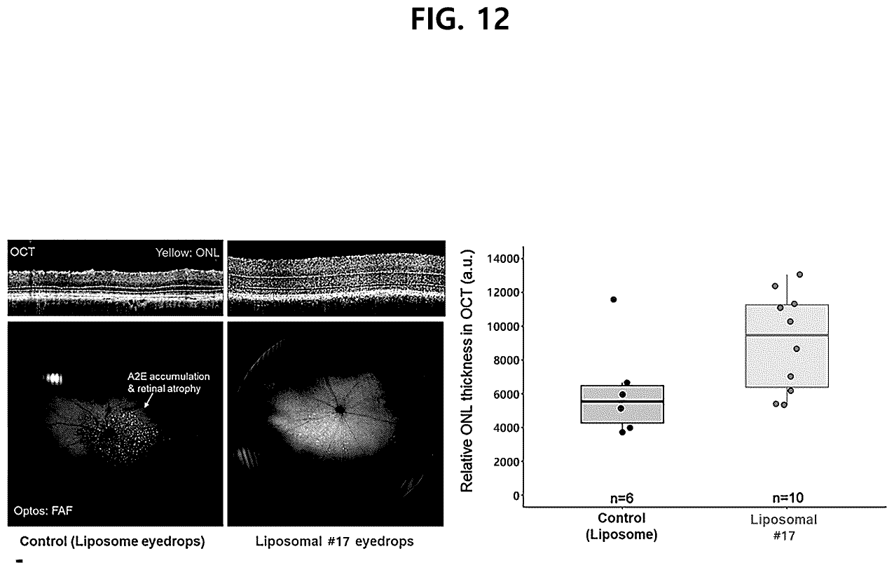

As shown in the OCT image analysis in FIG. 12, a clear difference was observed between the control group and the group treated with the compound #17 liposomal eye drops. In the control group, a pronounced overall reduction in ONL thickness and total retinal thickness was observed, indicating progressive atrophy of the photoreceptor layer. In contrast, the compound #17 liposomal eye drop-treated group exhibited relatively well-preserved ONL and total retinal structure, with attenuation of the thickness reduction. These findings suggest that compound #17 effectively suppresses structural degeneration of the photoreceptor layer.

Evaluation of A2E accumulation and retinal atrophy using FAF imaging further confirmed the protective effect of compound #17. In the control group, strong punctate autofluorescent signals were widely distributed, indicating pronounced deposition of A2E aggregates, and peripheral retinal atrophy was clearly evident. In sharp contrast, the compound #17 liposomal eye drop-treated group showed a significant reduction in excessive A2E fluorescence signals and a marked decrease in atrophic regions, demonstrating an overall preservation of retinal structure. These results indicate that compound #17 inhibits the pathological accumulation of A2E and alleviates the resulting retinal damage.

In addition, a box plot comparing relative ONL thickness calculated by OCT-based quantitative analysis showed that the compound #17-treated group (n=10) maintained higher ONL thickness compared with the control group (n=6).

Taken together, this example demonstrates that the liposome-formulated compound #17 eye drops are effectively delivered to the retina through topical administration and suppress A2E-associated photoreceptor layer damage while preserving retinal structural integrity.

### Example 9 Mode of Action (MOA)

The mode of action of compound #17 was identified as a dual mechanism that regulates two interconnected upstream axes to protect retinal pigment epithelium (RPE) cells. The first is the retinoid-oxidative stress regulatory axis (FABP5/HSPB1 axis), and the second is the transcriptional regulatory axis (PPAR/RXR axis). Through the coordinated modulation of these two axes, compound #17 blocks ATRL/A2E toxicity and effectively protects RPE cells.

### Alleviation of Retinoid-Oxidative Stress Via the FABP5/HSPB1 Axis

ABCA4/RDH8 double-knockout (Abca4/Rdh8−/−) mice were intraperitoneally injected with PBS or compound #17 (three times per week for 2 weeks), and 2 weeks after completion of dosing, the retina-RPE-choroid complex was collected for bulk RNA-seq analysis (n=3-4 per group) followed by DEG/volcano plot evaluation. Genes related to retinoid metabolism and oxidative stress were primarily examined. At the cellular level, oxidative stress was induced in RPE1 cells by H₂O₂ treatment (300 μM for 6 hours), followed by pretreatment with compound #17 (10 μM for 24 hours). Immunofluorescence staining and Western blot analysis were then performed to quantitatively assess HSPB1 protein expression and phosphorylation status (p-HSP27/total HSP27).

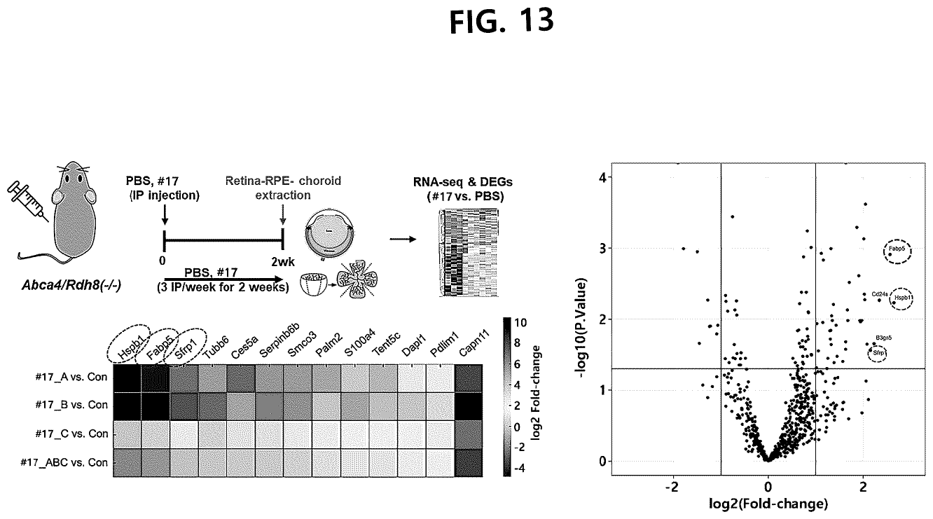

As shown in FIG. 13, FABP5, HSPB1, SFRP1 and other genes involved in retinoid metabolism and oxidative stress regulation were significantly upregulated in the compound #17-treated group. In particular, Fabp5 and Hspb1 exhibited statistically significant increases, confirming activation of the FABP5/HSPB1 axis. These gene expression changes indicate that compound #17 regulates the retinoid metabolic and oxidative stress defense systems at the transcriptional level.

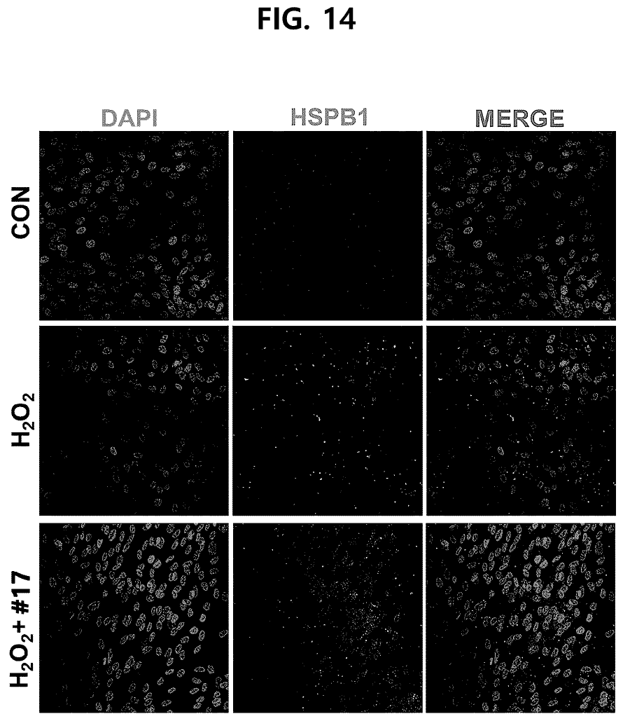

Furthermore, as shown in FIG. 14, HSPB1 fluorescence intensity in RPE1 cells was markedly reduced upon H₂O₂ treatment alone compared with the control, whereas co-treatment with compound #17 restored fluorescence intensity to a level comparable to that of the control. This demonstrates that compound #17 effectively prevents the reduction of HSPB1 protein expression caused by oxidative stress.

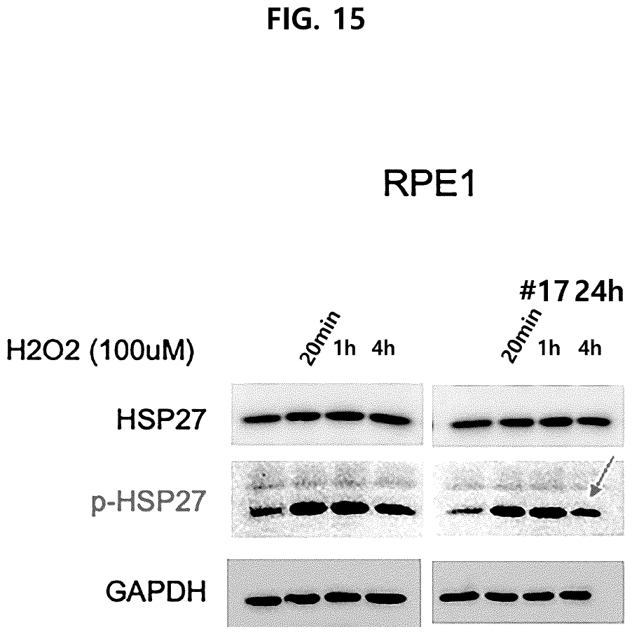

The phosphorylation state of HSPB1 has a direct impact on its chaperone activity. Excessive phosphorylation (increased p-HSPB1) leads to dissociation of large oligomeric structures, resulting in reduced chaperone function, whereas inhibition of phosphorylation (reduced p-HSPB1) promotes the formation of large oligomers, thereby enhancing chaperone stability and oxidative stress resistance. Western blot analysis confirmed suppression of excessive phosphorylation, as evidenced by decreased p-HSP27 levels (FIG. 15).

Collectively, these findings indicate that compound #17 not only restores HSPB1 protein expression but also optimizes its structural state to maintain chaperone activity, thereby conferring protection of RPE cells against retinoid toxicity and oxidative stress.

### Transcriptional Regulation Via the PPAR/RXR Axis

In this example, in silico screening and luciferase reporter assays were performed to identify the potential molecular targets of compound #17 and to determine its effect on nuclear receptor-mediated transcriptional activity. First, target candidates of compound #17 were predicted using SwissTargetPrediction analysis. The results suggested that compound #17 is highly likely to primarily target nuclear receptor family members, including estrogen receptor R (ESR2) and peroxisome proliferator-activated receptor α (PPARα). Additional potential targets were identified, including γ-butyrobetaine dioxygenase 1 (BBOX1), monoglyceride lipase (MGLL), monoamine oxidase B (MAOB), and cannabinoid receptor 2 (CNR2), which belong to the enzyme or G protein-coupled receptor classes.

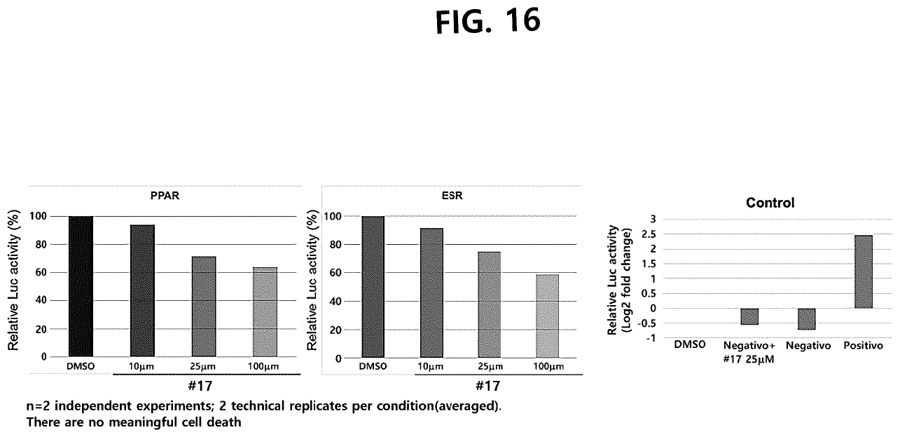

Subsequently, luciferase reporter assays were conducted to verify the effect of compound #17 on nuclear receptor-driven transcription. RPE1 cells expressing PPRE (Peroxisome Proliferator Response Element) or ERE (Estrogen Response Element) reporters were treated with compound #17 at concentrations of 0, 10, 25, and 100 μM, and luciferase activity was measured 24 hours after transfection. As shown in FIG. 16, luciferase activity driven by both PPAR and ESR reporters decreased in a dose-dependent manner following treatment with compound #17, with the greatest reduction observed in the 100 μM treatment group. Under the same experimental conditions, no significant reduction in cell viability was detected.

Taken together, these results indicate that compound #17 has the potential to bind nuclear receptors, particularly ESR2 and PPARα, and further functions to suppress PPRE- and ERE-mediated transcriptional activity. This mechanistic feature supports the therapeutic potential of compound #17 as a candidate capable of modulating pathological pathways governed by nuclear receptor signaling.

### Example 10 Safety Evaluation

### Basic Safety Assessment in Cells and Animals

To evaluate the basic safety of compound #17, cytotoxicity was examined by treating RPE cell lines with the compound at concentrations up to 500 μM. No significant toxicity was observed compared with the DMSO control group, indicating that compound #17 is well tolerated at high concentrations at the cellular level.

In addition, when mice were intraperitoneally administered compound #17 at 10 mg/kg three times per week for 2 months, no notable toxicity was detected, confirming the safety of repeated dosing in vivo.

### Single-Dose Toxicity Study

Single-dose toxicity of compound #17 was evaluated in C57BL/6 mice following a single intraperitoneal injection. Doses of 30, 100, 300, and 1000 mg/kg were administered to groups of 4, 4, 9, and 4 animals, respectively, and the control group received an equivalent volume of PBS. Mortality and behavioral changes were monitored immediately after dosing and for 3 days thereafter, and on day 3 all animals were sacrificed for gross and histopathological evaluation of major organs.

In the 30 and 100 mg/kg groups, no abnormal behaviors or adverse reactions were observed immediately after dosing, and no deaths occurred, resulting in a 100% survival rate. When 300 mg/kg—corresponding to 30-fold the efficacious dose of 10 mg/kg—was administered intraperitoneally, 8 of 9 animals showed no remarkable abnormal behaviors or adverse reactions, whereas 1 animal displayed reduced motility and 1 animal died. In the 1000 mg/kg group, transient reduction in activity was observed immediately after dosing in all 4 animals, 3 animals died, and 1 animal recovered to normal behavior within a few hours.

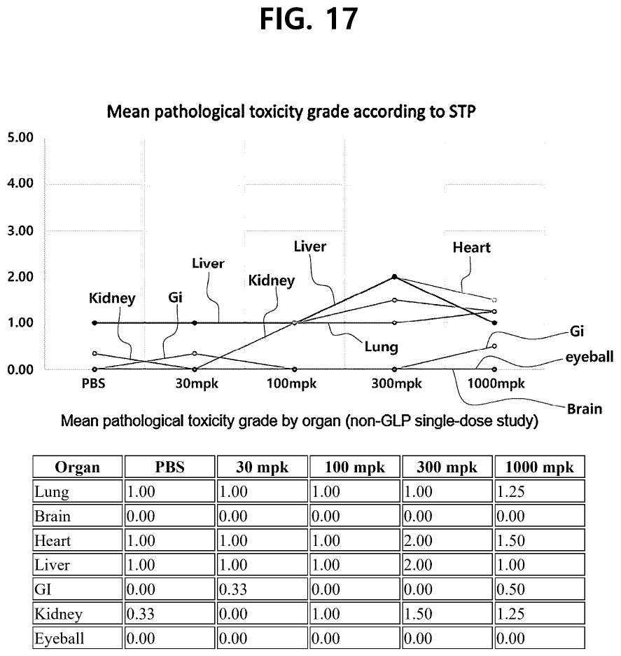

As shown in FIG. 17, histopathological analysis revealed no toxicological findings in the lung, brain, heart, gastrointestinal tract, or ocular tissues at any dose tested. Mild morphological changes were observed in the liver and kidney; however, no functional impairment markers were detected, and no toxicity grade ≥3 was identified. Based on these results, compound #17 is considered to exhibit a safety margin of at least 10-fold relative to its efficacious dose (10 mg/kg).

What is claimed is:

1. A method for preventing, ameliorating, or treating retinal diseases, comprising administrating an effective amount of one or more compounds represented by the following General Formulas 1 to 5: 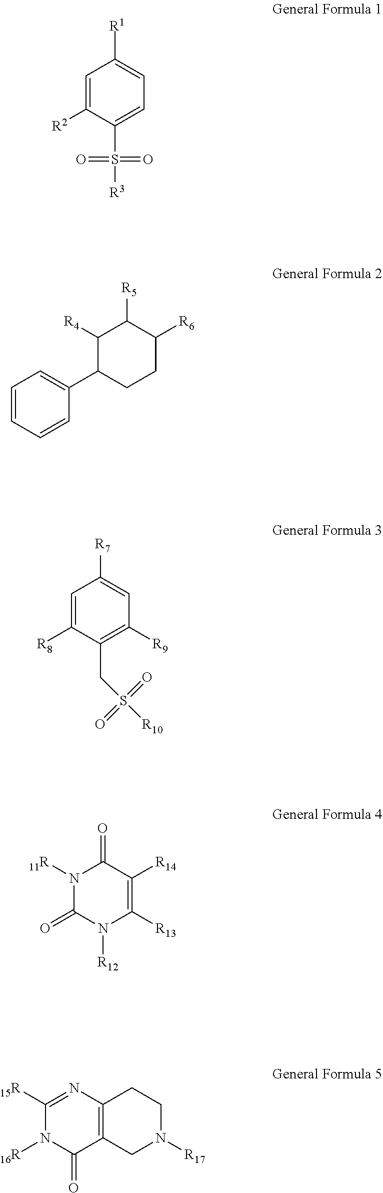
wherein R1 to R17 of General Formulas 1 to 5 are each a substituent selected from the group consisting of a hydrogen, a deuterium, a halogen atom, a hydroxyl group, a cyano group, an amino group, an amido group, a nitro group, a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, an alkoxy group, an aryloxy group, a silyl group, a substituted or unsubstituted (C2-C30) alkenyl group, a substituted or unsubstituted (C2-C30) cycloalkenyl group, a substituted or unsubstituted (C1-C30) heteroalkenyl group, a substituted or unsubstituted (C2-C30) alkynyl group, a substituted or unsubstituted (C6-C30) aryl group, a substituted or unsubstituted (C2-C30) heteroaryl group, an acyl group, a carboxylic acid, an ether, an ester, a nitrile, an isonitrile, a sulfanyl group, a sulfinyl group, a sulfonyl group, a phosphino group, and combinations thereof.

2. The method of claim 1, wherein R1 of General Formula 1 is a substituent selected from the group consisting of a hydrogen, a deuterium, a halogen atom, and combinations thereof,
R2 is a substituent selected from the group consisting of a hydroxyl group, a cyano group, an amino group, an amido group, a nitro group, an alkoxy group, an aryloxy group, a silyl group, an acyl group, a carboxylic acid group, an ether group, an ester group, a nitrile, an isonitrile, a sulfanyl group, a sulfinyl group, a sulfonyl group, a phosphino group, and combinations thereof, and
R3 is a substituent selected from the group consisting of a substituted or unsubstituted (C1-C30) heteroalkyl group, a substituted or unsubstituted (C2-C30) heterocycloalkyl group, a substituted or unsubstituted (C1-C30) heteroalkenyl group, a substituted or unsubstituted (C2-C30) heteroaryl group, and combinations thereof.

3. The method of claim 1, wherein R4 of General Formula 2 is a substituent selected from the group consisting of a hydrogen, a deuterium, a substituted or unsubstituted (C1-C30) alkyl group, and combinations thereof, and
R5 or R6 is each a substituent selected from the group consisting of a hydroxyl group, a cyano group, an amino group, an amido group, a nitro group, an alkoxy group, an aryloxy group, a silyl group, an acyl group, a carboxylic acid, an ether, an ester, a nitrile, an isonitrile, a sulfanyl group, a sulfinyl group, a sulfonyl group, a phosphino group, and combinations thereof.

4. The method of claim 1, wherein R7 to R9 of General Formula 3 are each a substituent selected from the group consisting of a hydrogen, a deuterium, a substituted or unsubstituted (C1-C30) alkyl group, and combinations thereof, and
R10 is a substituent selected from the group consisting of a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, a substituted or unsubstituted (C1-C30) heteroalkenyl group, a substituted or unsubstituted (C2-C30) heteroaryl group, and combinations thereof.

5. The method of claim 1, wherein R11 of General Formula 4 is a substituent selected from the group consisting of a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, a substituted or unsubstituted (C2-C30) alkenyl group, a substituted or unsubstituted (C2-C30) cycloalkenyl group, a substituted or unsubstituted (C1-C30) heteroalkenyl group, a substituted or unsubstituted (C2-C30) alkynyl group, a substituted or unsubstituted (C6-C30) aryl group, a substituted or unsubstituted (C2-C30) heteroaryl group, and combinations thereof,
R12 is a substituent selected from the group consisting of a hydrogen, a deuterium, a substituted or unsubstituted (C1-C30) alkyl group, and combinations thereof, and
R13 and R14 are fused to form a substituted or unsubstituted ring.

6. The method of claim 1, wherein R15 of General Formula 5 is a substituent selected from the group consisting of a substituted or unsubstituted (C1-C30) alkyl group, a substituted or unsubstituted (C3-C30) cycloalkyl group, a substituted or unsubstituted (C1-C30) heteroalkyl group, a substituted or unsubstituted (C2-C30) heterocycloalkyl group, a substituted or unsubstituted (C6-C30) aryl (C1-C30) alkyl group, a substituted or unsubstituted (C1-C30) heteroalkenyl group, a substituted or unsubstituted (C2-C30) heteroaryl group, and combinations thereof,
R16 is a substituent selected from the group consisting of a hydrogen, a deuterium, a substituted or unsubstituted (C1-C30) alkyl group, and combinations thereof, and
R17 is a substituent selected from the group consisting of a substituted or unsubstituted (C1-C30) alkyl group, a substituted or unsubstituted (C3-C30) cycloalkyl group, a (C1-C30) heteroalkyl group, a substituted or unsubstituted (C2-C30) heterocycloalkyl group, an amino group, an amido group, a nitro group, a carboxylic acid, an ester, and combinations thereof.

7. The method of claim 1, wherein the retinal disease is one or more selected from the group consisting of hereditary macular degeneration, age-related macular degeneration, myopic macular degeneration, and macular degeneration caused by inflammation.

8. The method of claim 7, wherein the hereditary macular degeneration is one or more selected from the group consisting of retinitis pigmentosa, vitelliform macular dystrophy, Stargardt disease, X-linked retinoschisis, and cone dystrophy.

9. The method of claim 7, wherein the macular degeneration caused by inflammation is macular degeneration caused by Behcet's uveitis

10. The method of claim 1, wherein the compound is administered at a concentration of 0.1 μM to 1000 μM.

11. The method of claim 1, wherein the compound has one or more effects of cell death inhibition, cell aging inhibition, or intracellular reactive oxygen species inhibition.
